(19) United States
(12) Patent Application Publication
(10) Pub. No.: US 2025/0049413 A1
(43) Pub. Date: Feb. 13, 2025

(54) HEALTH DIAGNOSTIC SYSTEM AND A METHOD FOR ANALYZING THE HEALTH OF AN ANIMAL

(71) Applicant: Laboratory of Data Discovery for Health Limited, Pak Shek Kok (HK)

(72) Inventors: Joshua Wing Kei Ho, Pak Shek Kok (HK); Shichao Ma, Pak Shek Kok (HK)

(21) Appl. No.: 18/446,614

(22) Filed: Aug. 9, 2023

(51) Int. Cl. A61B 7/04; G16H 50/20; G16H 50/30; H04R 1/46

(52) U.S. Cl. CPC A61B 7/04; G16H 50/20; G16H 50/30; H04R 1/46

(57) ABSTRACT

A health diagnostic system and a method for analyzing health of an animal. The system includes a signal receiver module arranged to receive a record of sound generated by an organ in a body of an animal when the organ performs a predetermined function for a predetermined period; a signal denoising module arranged to reduce a noise signal in the record; and a health diagnostic analyzing module arranged to analyze a health issue of the animal based on normal sounds generated by the organ and adventitious sounds in the denoised record.

### TECHNICAL FIELD

The invention relates to a health diagnostic system and a method for analyzing the health of an animal, and particularly, although not exclusively, to a machine-learning-based health diagnostic system.

### BACKGROUND

Stethoscopes have been an essential tool for medical professionals for years. They are used by doctors, veterinary surgeons and other healthcare practitioners to listen to various sounds within the body during physical exams. Doctors and veterinary surgeons may use stethoscopes to listen to the heart and lungs of a patient (who may be a human or non-human patient), which can help in detecting abnormal sounds, murmurs, arrythmia, and respiratory problems. Stethoscopes can also be used to monitor blood flow and blood pressure in arteries and veins.

Stethoscopes may also be used to listen to bowel sounds in the abdomen to detect digestive issues. They can also be used to detect fluid buildup in the lungs or around the heart, as well as sounds of the gastrointestinal tract to detect issues like constipation, diarrhea, or bloating. To facilitate recording of these sounds generated by organs within the body, digital stethoscopes may be used if necessary.

### SUMMARY OF THE INVENTION

In accordance with a first aspect of the present invention, there is provided a health diagnostic system comprising: a signal receiver module arranged to receive a record of sound generated by an organ in a body of an animal when the organ performs a predetermined function for a predetermined period; a signal denoising module arranged to reduce a noise signal in the record; and a health diagnostic analyzing module arranged to analyze a health issue of the animal based on normal sounds and adventitious sounds generated by the organ in the denoised record.

In accordance with the first aspect, wherein the adventitious sounds include murmur or an arrythmia sound pattern generated by the heart of the animal or abnormal sounds generated by other organs of the animal.

In accordance with the first aspect, the health diagnostic analyzing module includes a neural network arranged to classify the record so as to mark at least one health indicator associated with the health issue.

In accordance with the first aspect, the denoising module is arranged to reduce a disturbance caused by the noise signal in the record so as to increase a prediction accuracy of the health issue provided by the health diagnostic analysing module.

In accordance with the first aspect, the denoising module is arranged to transform the record with discrete wavelet transform (DWT) to reduce a noise signal in the record.

In accordance with the first aspect, after decomposition of signals in the record, thresholding coefficients is not performed, instead a resampling processed is performed to obtain the denoised signal.

In accordance with the first aspect, the denoised signal is resampled to 48 KHz and the frequency range of the signals in the record less than 300 Hz is retained.

In accordance with the first aspect, the neural network includes a lightweight neural network arranged to run on a computing device with a mobile or lightweight processor.

In accordance with the first aspect, the signal receiver module is further arranged to validate the normal sounds generated by one or more organs in the body of the animal for further process.

In accordance with the first aspect, the signal receiver module comprises a signal recognition module arranged to validate the normal sounds by recognizing an existence of the sounds in the record received by the signal receiver module.

In accordance with the first aspect, the signal denoising module and the health diagnostic analyzing module are arranged to process the record of sound upon successful validation of normal sounds in the record.

In accordance with the first aspect, the record includes one or more clips extracted from the record received by the signal receiver module.

In accordance with the first aspect, the signal receiver module is further arranged to prolong the record of sound for further process upon successful validation of the normal sounds.

In accordance with the first aspect, the successful validation is indicated by positive classification of two consecutive clips, each containing the normal sounds.

In accordance with the first aspect, the health issue includes a risk of valvular heart disease (VHD).

In accordance with the first aspect, the neural network comprises a downstream phonocardiogram (PCG) classification network for VHD screening.

In accordance with the first aspect, the downstream PCG classification network is trained based on an upstream self-supervised learning network for PCG classification and a transfer learning process.

In accordance with the first aspect, the health diagnostic analyzing module is arranged to label a phonocardiogram associated with the record received by the signal receiver, thereby facilitating the downstream PCG classification network to mark the at least one health indicator associated with the health issue.

In accordance with the first aspect, the record of sound includes heartbeats, breathing sound, sound of lung or sound of bowel movement.

In accordance with the first aspect, the signal receiver module comprises a microphone arranged to generate the record of sound generated by one or more organs in the animal.

In accordance with the first aspect, the system may be deployed and hosted in consumer-grade smartphones, digital devices, computers or the cloud.

In accordance with a second aspect of the present invention, there is provided a method for analyzing health of an animal, comprising the steps of: receiving a record of sound generated by an organ in a body of the animal when the organ performs a predetermined function for a predetermined period; reducing a noise signal in the record; and analyzing a health issue of the animal based on normal sounds and adventitious sounds in the denoised record.

In accordance with the second aspect, wherein the adventitious sounds include murmur or an arrythmia sound pattern generated by the heart of the animal or abnormal sounds generated by other organs of the animal.

In accordance with the second aspect, the step of analyzing the health issue of the animal based on normal sounds and murmur or an arrythmia sound pattern in the denoised record includes classify the record by a neural network so as to mark at least one health indicator associated with the health issue.

In accordance with the second aspect, the step of reducing a noise signal in the record is performed to reduce a disturbance caused by the noise signal in the record so as to increase a prediction accuracy of the health issue being analyzed.

In accordance with the second aspect, the method further comprises the step of validating the normal sounds generated by one or more organs in the body of the animal for further process, by recognizing an existence of the sounds in the record received, prior to the steps reducing a noise signal in the record and the step of analyzing a health issue of the animal based on normal sounds and adventitious sounds generated by the organ in the denoised record.

In accordance with the second aspect, the step of reducing a noise signal in the record includes transforming the record with discrete wavelet transform (DWT) to reduce a noise signal in the record.

In accordance with the second aspect, after decomposition of signals in the record, the step of transforming the record with discrete wavelet transform (DWT) is performed without thresholding coefficients, instead a resampling processed is performed to obtain the denoised signal.

In accordance with the second aspect, the denoised signal is resampled to 48 KHz and the frequency range of the signals in the record less than 300 Hz is retained.

In accordance with the second aspect, the neural network includes a lightweight neural network arranged to run on a computing device with a mobile or lightweight processor.

In accordance with the second aspect, the health issue includes a risk of valvular heart disease (VHD).

In accordance with the second aspect, the neural network comprises a downstream PCG classification network for VHD screening.

In accordance with the second aspect, the downstream PCG classification network is trained based on an upstream self-supervised learning network for PCG classification and a transfer learning process.

In accordance with the second aspect, the step of analyzing the health issue of the animal based on normal sounds and adventitious sounds in the denoised record comprising the step of labelling a phonocardiogram associated with the record received by the signal receiver, thereby facilitating the downstream PCG classification network to mark the at least one health indicator associated with the health issue.

In accordance with the second aspect, the record of sound includes heartbeats, breathing sound, sound of lung or sound of bowel movement.

In accordance with the second aspect, the method further comprises the step of generating the record of sound generated by one or more organs in the animal recorded by a microphone.

### BRIEF DESCRIPTION OF THE DRAWINGS

Embodiments of the present invention will now be described, by way of example, with reference to the accompanying drawings in which:

FIG. 1 is a schematic diagram of a computer server which is arranged to be implemented as a health diagnostic system in accordance with an embodiment of the present invention.

FIG. 2 is a block diagram showing a health diagnostic system in accordance with an embodiment of the present invention.

FIG. 3 is a flow diagram showing a denoising process of the received record.

FIG. 4 is an illustration showing architectures of (a) the upstream model and (b) downstream model. The parameters in the first convolutional layers from the upstream model were transferred to the downstream model and frozen after the upstream model converged in all six tasks.

FIG. 5 is an illustration showing the visualization of six kinds of transformations which were applied to the PCG data. Pseudo-labels were automatically generated after the application of transformations.

FIG. 6 shows a random length of portion at the beginning of the original signal is cropped and attached to the tail of the signal. The perturbated signal starts from a random position of a cardiac cycle instead of the beginning of S1. The purpose of perturbation is to further test the classification model's robustness.

FIG. 7 is an illustration showing the flow of developing the mobile application prototype. A screenshot of the application is also included. Users can press corresponding buttons to fine-tune the pre-trained model or make inferences locally. The time required to accomplish a specific task is recorded and presented on the interface.

FIG. 8A is a box chart plot the aggregated result. The model can perform significantly better when pre-trained with unlabeled data in the upstream, whether the signals start from S1 or not.

FIG. 8B is another box chart plot the aggregated result of a different attempt.

FIG. 9 is a plot showing the visualization of the upstream model's training history shows that the model will eventually converge in all six tasks. Task 1 is the easiest to learn, while the third and sixth tasks are relatively tricky.

FIG. 10 is a confusion matrix for a, the aggregated cross-validation result of the median attempt from the model without the SSL on the edited dataset, and b, the aggregated cross-validation result of the median attempt from the model with the SSL on the edited dataset.

FIG. 11 shows line charts plot (a) the aggregated result of two models' attempts with an increasing noise level. The model with SSL shows more robustness when gaussian noise is injected into the signals. (b) the performance of two methods when different numbers of training epochs are conducted.

FIG. 12A is the class activation maps of both the actual category and predicted category, for sample no. 17. The samples were misclassified by the model without SSL but correctly classified by the model with SSL.

FIG. 12B is the class activation maps for sample no. 46.

### DETAILED DESCRIPTION OF THE PREFERRED EMBODIMENTS

The inventors devised that cardiovascular disease diseases (CVDs) consist of diseases that are a primary cause of illness and death worldwide. Valvular heart disease (VHD) is a type of CVD in which one or more heart valves has a functional or structural deficit that impacts blood flow in the heart.

Undetected and untreated VHD can lead to deterioration of cardiac function and mortality. It remains a major health burden in developed countries. Detection of abnormal heart sounds (cardiac murmurs) may be a simple strategy for the population VHD screening. With the widespread availability of mobile smartphones, phonocardiogram (PCG) data can be collected and analyzed on these edge devices for VHD screening.

For example, interpretable PCG in the database contains four recognizable heart sound stages (S1,systole, S2, and diastole). The presence of different patterns of murmur in the sound segment may indicate VHD. Heart sounds from the dataset may be classified into normal and abnormal. Alternatively, PCG dataset may contain five categories (mitral valve prolapse, mitral stenosis, mitral regurgitation, aortic regurgitation, and normal heart sound), and each class may have 200 clean PCG samples.

In addition, PCG data may be applied to machine learning-based heart sound classification models for VHD screening. Heart sound classification models may be grouped into artificial neural network-based, support vector machine-based, hidden Markov model-based, and clustering-based methods.

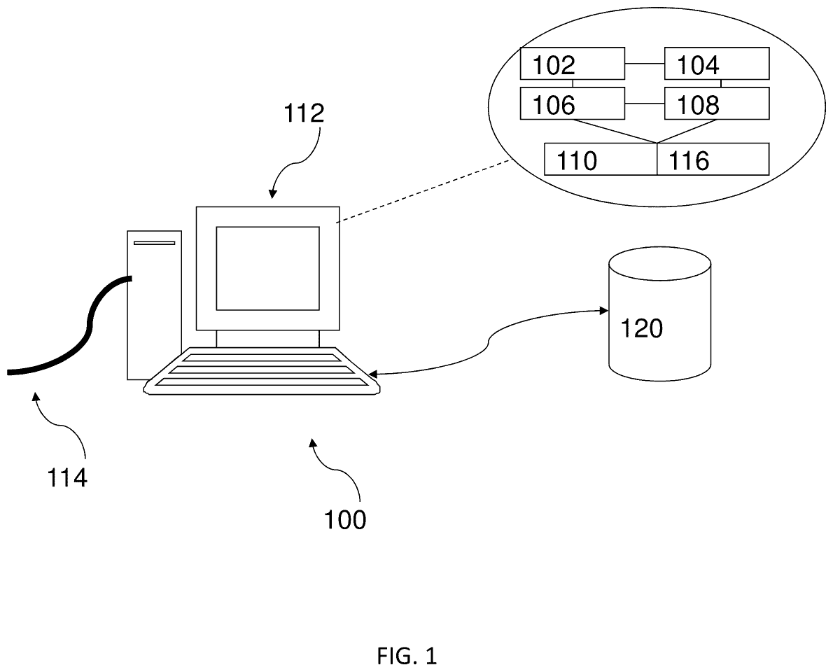

Referring to FIG. 1, an embodiment of the present invention is illustrated. This embodiment is arranged to provide a health diagnostic system comprising a signal receiver module arranged to receive a record of sound generated by an organ in a body of an animal when the organ performs a predetermined function for a predetermined period; a signal denoising module arranged to reduce a noise signal in the record; and a health diagnostic analyzing module arranged to analyze a health issue of the animal based on normal sounds and adventitious sounds generated by the organ, such as murmur generated by the heart, in the denoised record. For clarity, the term “animal” should be considered as having the biological definition, and therefore includes human beings, as well as non-human animals such as, but not limited to, pets, cattle or exotic wildlife.

In this example embodiment, the interface and processor are implemented by a computer having an appropriate user interface. The computer may be implemented by any computing architecture, including portable computers, tablet computers, stand-alone Personal Computers (PCs), smart devices, Internet of Things (IOT) devices, edge computing devices, client/server architecture, “dumb” terminal/mainframe architecture, cloud-computing based architecture, or any other appropriate architecture. The computing device may be appropriately programmed to implement the invention.

The system may be used to provide information related to health of a patient (e.g., a human being) based on an analysis of biomedical sound generated by certain organ, such as the heart of the patient. For example, a record of sound clip mixed with “normal” heartbeat and heart murmur, which is an unusual sound that is detected during a heartbeat when the blood flow is not smooth, may be provided to the system for determination of a health issue of a patient. These abnormal sounds, i.e., murmur or an arrythmia sound pattern, can be caused by various factors including valve problems, congenital heart defects, or infections. However, not all murmurs are harmful and some may not require medical attention, therefore labelling them accurately may help a medical practitioner to take necessary measures towards different murmurs being identified.

It may be crucial to have a healthcare provider evaluate the severity and cause of the murmur to determine if it is benign or if it is an indicator of a more serious underlying condition. Treatment options will vary depending on the cause of the murmur and the severity of the condition. In some cases, medications or surgical procedures may be necessary to correct the underlying issue and prevent further complications.

For example, healthcare providers may also use other diagnostic tools, such as echocardiograms, electrocardiograms (ECGs), or chest X-rays, to verify the health issues in concern. These tests can help to visualize the structure and function of the heart, and provide more information about the underlying cause of the murmur or the arrythmia sound pattern.

Without deviating from the spirit of the present invention, recording of sounds generated by any animal species other than human being may also be processed by the system of the present invention, and may also help veterinary practitioners to quickly analyze any health issue of an animal. In addition, sounds generated by other organs, and multiple organs may be analyzed using the system.

As shown in FIG. 1 there is a shown a schematic diagram of a computer system or computer server 100 which is arranged to be implemented as an example embodiment of a system comprising computer-executable instructions, when being executed, arranged to perform the abovementioned method for analyzing health of an animal. In this embodiment the system comprises a server 100 which includes suitable components necessary to receive, store and execute appropriate computer instructions. The components may include a processing unit 102, including Central Processing Unit (CPUs), Math Co-Processing Unit (Math Processor), Graphic Processing Unit (GPUs) or Tensor processing Unit (TPUs) for tensor or multi-dimensional array calculations or manipulation operations, read-only memory (ROM) 104, random access memory (RAM) 106, and input/output devices such as disk drives 108, input devices 110 such as an Ethernet port, a USB port, etc. Display 112 such as a liquid crystal display, a light emitting display or any other suitable display and communications links 114. The server 100 may include instructions that may be included in ROM 104, RAM 106 or disk drives 108 and may be executed by the processing unit 102. There may be provided a plurality of communication links 114 which may variously connect to one or more computing devices such as a server, personal computers, terminals, wireless or handheld computing devices, Internet of Things (IoT) devices, smart devices, edge computing devices. At least one of a plurality of communications link may be connected to an external computing network through a telephone line or other type of communications link.

The server 100 may include storage devices such as a disk drive 108 which may encompass solid state drives, hard disk drives, optical drives, magnetic tape drives or remote or cloud-based storage devices. The server 100 may use a single disk drive or multiple disk drives, or a remote storage service 120. The server 100 may also have a suitable operating system 116 which resides on the disk drive or in the ROM of the server 100.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

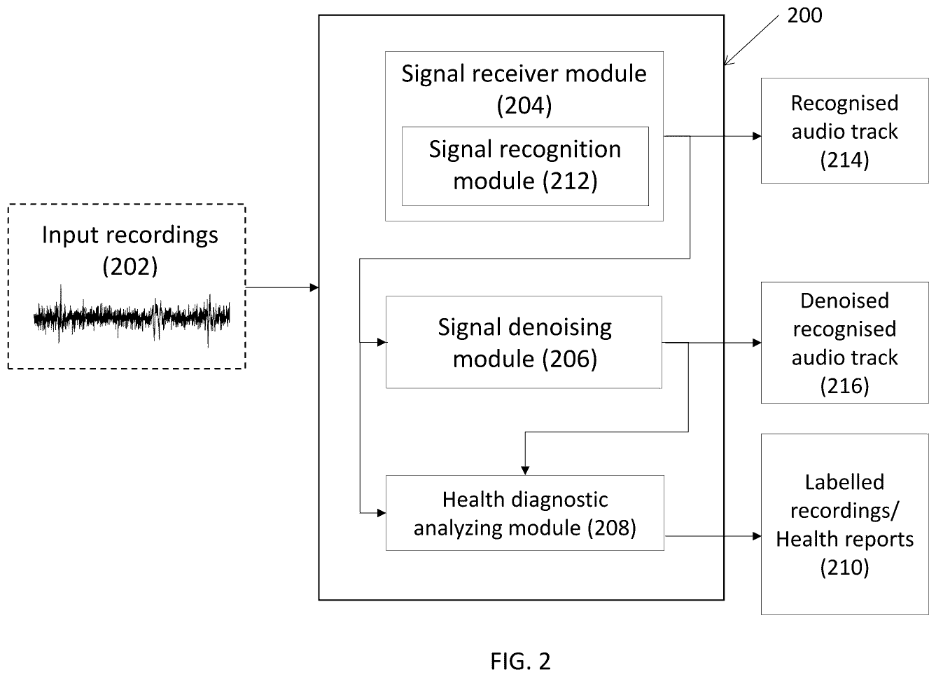

With reference to FIG. 2, there is provided an embodiment of a health diagnostic system 200. In this embodiment, the server 100 is used as part of a system as arranged to receive a record of sound 202 or sound clip generated by an organ, such as the heart of a patient, and after processing of the sound clip, generating a report listing health issues of the patient based on the patterns of the periodical sounds and murmur being recorded, which help medical practitioners to follow up with other diagnostic tests if necessary, to determine if the patient is suffering from any CVD, such as valvular heart disease.

In this example, the system 200 comprises a signal receiver module 204, such as microphone for detecting soundwaves which may be further recorded in a memory device, or it may be a software module arrange to read a pre-recorded sound clip in form or a digital file of electronic form. For example, a recording of heartbeat of a patient may be provided to the system for analysis.

Without wishing to be bound by theory, heart sound recordings may be categorized as “unsure”, which indicates that the recording was too noisy to be interpretable, therefore recordings may affect the performance or accuracy of the analysis. In this example, the health diagnostic system 200 comprises a signal denoising module 206 arranged to reduce a noise signal in the record 202, which enable the health diagnostic analyzing module 208 to analyze a health issue based on records with a clearer signal pattern. For example, a digital signal processor (DSP) may be used to process a noisy sound clip to specifically separate the noise signal from the sounds generated by an organ in a body of an animal when the organ performs a predetermined function for a predetermined period. Alternatively, the denoising process may be performed by utilizing machine learning or a neural network processing engine.

In addition, the denoising module 206 may reduce a disturbance caused by the noise signal in the record so as to increase a prediction accuracy of the health issue provided by the health diagnostic analysing module. By denoising the raw sound clip provided to the system 200, processing resources in a later stage, such as computational, memory and transmission resources, required for operating the health diagnostic analyzing module 208 may be reduced. Preferably, the health diagnostic analyzing module 208 includes a neural network arranged to classify the record so as to mark at least one health indicator associated with the health issue, and the neural network may be a lightweight neural network arranged to run on a computing device with a mobile or lightweight processor, such as a processor included in a smartphone, a tablet computer, a handheld computing device or the like. Preferably, the denoising module 206 may also output a denoised recognized audio track 216, for further processing, e.g. by the health diagnostic analyzing module, or the output audio track 214 may be saved as a data file for future use or analysis.

Preferably, the denoising module 206 may denoise the input recording 202 by employing Wavelet-based technique, such as discrete wavelet transform (DWT), to reduce noises in heart sound recordings (or recordings of other types of biomedical sound). For example, thresholding may be applied to the coefficients output from the decomposition and the parameter selections on both the DWT and the thresholding techniques are compared, although other methods may be used to implement the denoising module 206, including the use of machine learning networks that have been trained to identify certain types of signals for filtering or selection.

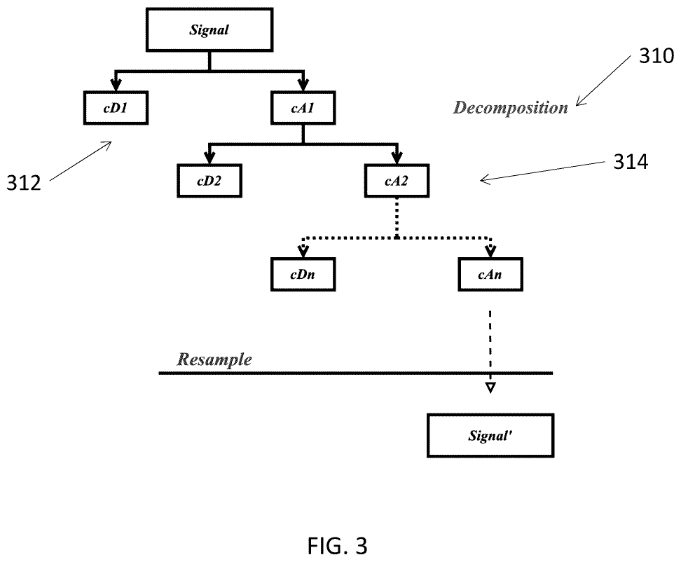

Alternatively, thresholding coefficients after decomposition 310 may not be necessary. For example, with reference to FIG. 3, all detailed coefficients 312 may be zeroed and the approximation coefficients 314 may be retained at the highest level. Then the reconstruction of the signal may be conducted based on the approximation coefficients 314, in a resampling process. By employing this improved method, the exclusion of the thresholding procedure and its replacements by simply wiping high-frequency signals would significantly reduce the time complexity, which may enable the execution of the algorithm in devices with the limited computational resource.

Preferably, providing a denoised signal may help reducing a disturbance caused by the noise signal in the record so as to increase a prediction accuracy of the health issue provided by the health diagnostic analysing module.

To further improve the accuracy of the system, referring to FIG. 2, the signal receiver module comprises a signal recognition module 212 for “screening” out the invalid signals or recordings for further processing. Preferably, the signal receiver module may further validate the sounds generated by one or more organs in the body of the animal for further process, for example, by recognizing an existence of the sounds generated by one or more organs in the record received using the signal recognition module. In some exemplary embodiments of the present invention, normal sounds are heart sounds which are periodical sounds in high frequencies, and preferably by recognizing the periodical pattern and the frequencies of the input periodical sounds, signal recognition may immediately indicate whether the heartbeat is successfully captured to validate the input sounds. On the other hand, abnormal sounds (or adventitious sounds) may include (but not limited to) systolic or diastolic murmurs in heart sound, and crackles, wheezes and rhonchi.

Preferably, the signal denoising module and the health diagnostic analyzing module are arranged to process the record of sound upon successful validation of normal sounds in the record. For example, the signal receiver module may only choose to pass the recorded signals to for further processing only after successfully validating an existence of heart sounds in the recording, and preferably a clear record of heartbeats that may be processed by a light weight processor in a smartphone. Otherwise, the system may provide an error message to notify the user to reposition the microphone a correct position so as to capture a record that may be processed by the denoising module and the health diagnostic analyzing module implemented by using the smartphone processor, after successful validation of the sounds. Advantageously, the accuracy of the analysis may be enhanced by ensuring that records that may be blurry or captured at incorrect positions of the patient body are not further processed or analyzed.

By performing an additional signal recognition process, an immediate indication of whether the heartbeat is successfully captured, e.g., by a built-in microphone in a smartphone, may be provided so that the people who perform the self-auscultation can be informed and the recording of PCG data can start with the assurance that valid information is involved. Preferably, the small-footprint keyword spotting system widely used in virtual voice assistants may be employed, the classifier may be constructed in a similar way to make the system accurate, responsive and can be run with low power consumption. Referring to FIG. 2, the signal receiver module is arranged to output a recognized audio track 214 for further processing, e.g. by a signal denoising module or the health diagnostic analyzing module, or the output audio track 214 may be saved as a data file for future use or analysis.

Preferably, the input record may include one or more one-second clip extracted from the record received by the signal receiver module, and the signal receiver module may further prolong the record of sound for further process, e.g., classification of diseases or risks, upon successful validation of the sounds generated by one or more organs in the input records.

For example, heart sound recordings may be windowed into 1-second clips non-overlappingly, and the vast majority of clips would contain a heartbeat because the normal range of resting heart rate is from 60 to 100 bpm. The 1-second window interval will also expedite the procedure of determining whether a heartbeat is captured compared with a longer interval. Furthermore, the input clips may be downsampled to 1K Hz to ensure the inclusion of the heart sound frequency range less than 300 Hz and reduce the complexity of input data for CNN. Heart sound clips were scaled to the range between 0 and 1.

Preferably, the recognition module may be trained based on a CNN tuned with labelled data. Training heart sound datasets, with samples that were negatively labelled included unrecognized heart sound recordings. In addition, white noises with small amplitude and blank recordings were generated to mimic the scenario that the smartphone is placed in a quiet environment and the microphone captures no heartbeat. Positively labelled data including both normal and abnormal heart sound recordings with good quality may also be included in the training process.

In one example embodiment, the CNN was constructed to be as lightweight as possible so that it will later take up as little computing resources and storage space as possible in the mobile device. The input layer of CNN was built to accept a 1D heart sound signal which contains 1000 samples. The signal may then be processed with three convolution-max pool layers. The kernel size of the three convolutional layers was 100, 50 and 10, respectively, and the kernel size of the max-pooling layers was 4. The number of filters for each of the three convolutional layers was 16, 8 and 4. Then the last pooling layer was followed by three fully connected dense layers with a descending number of units. Eventually, the output layer with only one node representing the result of binary classification was added at the tail of the network. ReLU activation function was adopted in all hidden layers, while the sigmoid function was used in the output layer. The training loss was calculated as binary cross-entropy between the actual and desired output. The network was trained with the Adam optimizer, and 10% of training data was randomly split out as the validation set to enable the early stop of the training process and save the model with the highest validation accuracy.

Optionally, a simple fault-tolerance mechanism may be adopted in an embodiment of the heartbeat recognizer. For example, the successful validation is indicated by positive classification of two consecutive one-second clips each contains the sounds generated by organs in the body, thus the recording of heart sounds will start after two consecutive 1-second clips are positively classified to avoid the situation that a heartbeat-like noise starts the recording process by verifying the periodicity of the sound. Similarly, the recording process will halt after two consecutive 1-second clips are negatively classified to ensure that the process will not be interrupted by bradycardia.

In one example embodiment a lightweight 1D convolution neural network (CNN) architecture is used for analyzing a health issue of the animal based on sounds generated by organs in the body and adventitious sounds, such as murmur, an arrythmia sound pattern or irregular heartbeats, in the denoised record. An example 1D CNN were constructed by following the strategies below to minimize the size of the network and maintain precision simultaneously. Upon completion of analysis, output such as labelled recordings or even automatically generated health report 210 may be provided.

Firstly, complicated components were avoided, including residual blocks, extractors and enhancers in the neural network. Those components were invented to achieve certain objectives. For instance, residual blocks were initially proposed to tackle gradient-relevant problems in the backpropagation stage of extremely deep neural networks. This component proved to be helpful in tasks of processing complex and high-dimensional data with giant models. However, a relatively shallow network with only essential layers of CNN is enough to classify PCG data as a periodic signal, especially when the data is clean.

Secondly, small-sized convolutional filters were applied in all convolutional layers. As suggested in the SqueezeNet, the number of parameters will be largely reduced by adopting small filters under the budget of a certain number of filters.

Thirdly, features outputted by the convolutional layers were max-pooled so that fewer parameters in the following fully connected dense layers would be enough to process the features. It is devised by the inventors that, machine learning classifiers could achieve high accuracy on a feature vector of length around 40, which may be distilled from raw PCG data. The output of the convolutional and pooling layers was condensed enough before feeding it into the dense layers to reduce the need for parameters.

In one preferred embodiment, the neural network comprises a downstream PCG classification network, which may be used for screening a risk or potential of valvular heart disease (VHD). For example, the health diagnostic analyzing module, upon receiving the denoised record associated with a phonocardiogram being generated, the PCG classification network may label a phonocardiogram received by the signal receiver, thereby facilitating the downstream PCG classification network to mark the at least one health indicator associated with the health issue.

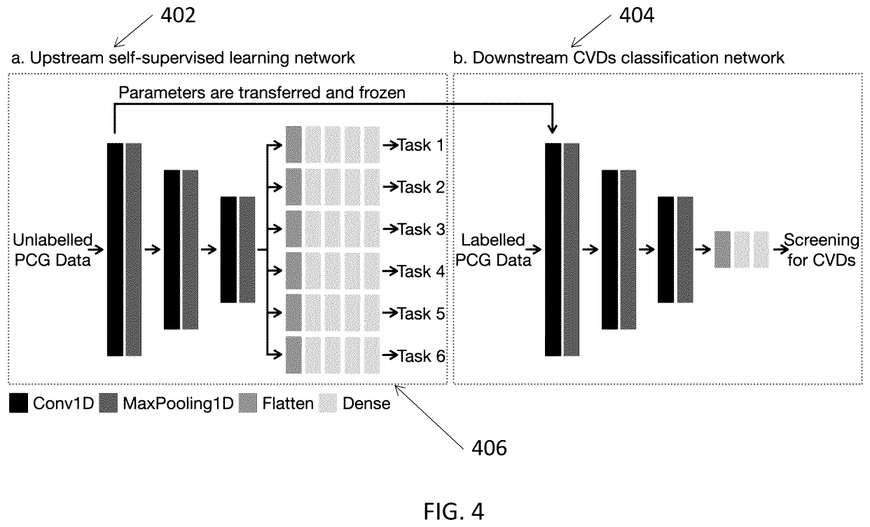

For example, referring to FIGS. 4 to 6, there is shown an example embodiment of the operation of the system which a user may record sounds of his chest which include sounds generated by the heart, and upload the recorded sound clip to the system for VHD screening, for example via an application installed on a smartphone or tablet device. Preferably, the screening process may be locally performed by the built-in processor of the smartphone or tablet device, which may be a lightweight processor or a processor with comparatively limited resources, e.g., processing power and/or bandwidth, then that in a computer server, thus it may be preferable to slim down the neural network that may be specifically designed to run on mobile devices, or edge devices.

Preferably, the downstream PCG classification network is trained based on an upstream self-supervised learning network for PCG classification and transfer learning. With reference to FIG. 4, the network's input layer was employed at the beginning of the model to accept the 1D PCG signal containing 2000 samples. The signal was then processed with three convolution-max pool layers. The size of kernels in all three convolutional layers was 20, while the kernel size of all three pooling layers was 4. The number of filters for each of the three convolutional layers was 16, 8 and 4, respectively. The last pooling layer was followed by one fully connected dense layer with 128 units. Eventually, the output layer with five nodes outputs the result of a 5-class categorical classification through a SoftMax activation function. Tanh activation function was adopted in all hidden layers. The training loss was calculated as the categorical cross-entropy between the actual and target output. Adam algorithm may be adopted during the optimization of the network. In this example, the entire model only contains 20,193 parameters.

The forward propagation of 1D CNN can be symbolically expressed as:

$$Y = F ( X \vert Θ ) = {f}_{n} ( … {f}_{2} ( {f}_{1} ( X \vert {θ}_{1} ) \vert {θ}_{2} ) \vert {θ}_{n} ) \qquad ( 1 )$$

In this equation, Y represents the predictions of CNN, X is the input 1D PCG data, and fₙ denotes the computation on the nₜₕ layer where θₜₕ stands for the learnable parameters on that layer. Moreover, the convolutional layer's operation can be described as follows:

$${Y}_{n} = {f}_{n} ( {X}_{n} \vert {Θ}_{n} ) = ϕ ( W ⊗ {X}_{n} + b ) , {θ}_{n} [ W , b ] \qquad ( 2 )$$

Here, φ is the activation function applied to the layer, ⊗ is the convolution operation, W denotes a collection of 1D convolutional receptive fields implemented to extract features, and b is the bias vector. Yₙ and Xₙ are the current layer's output and the previous layer's output, respectively.

In this example, an upstream task (also known as a pretext task) was designed to tune the parameters in the upstream network to recognize important representations from data. Then the tuned parameters can be transferred to a downstream network to assist the downstream task.

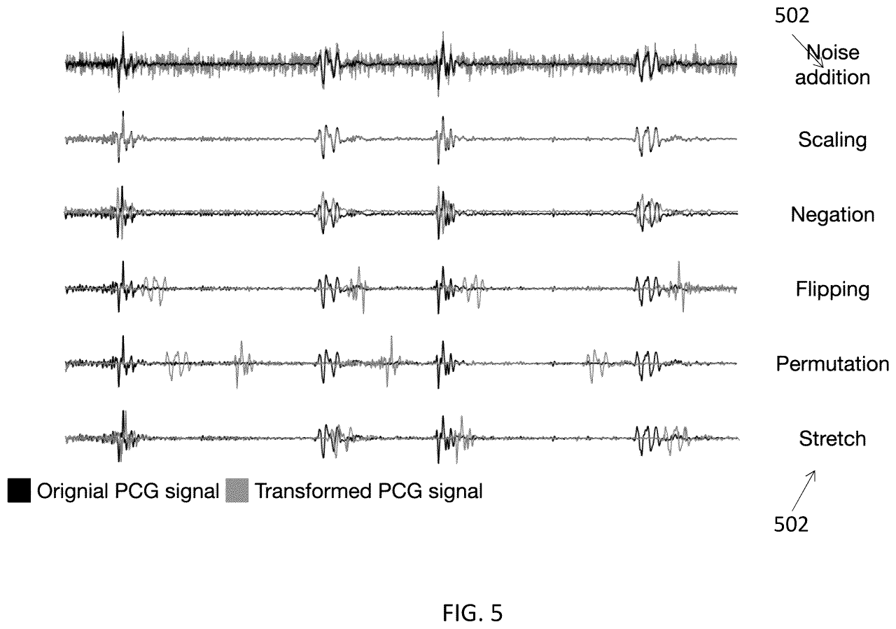

Preferably, referring to FIG. 5, six different transformations 502 may be applied to the unlabeled PCG samples 502 to produce the transformed dataset Xₜ and six sets of pseudo-labels. In one preferred embodiment, a 1D CNN as an upstream model in a multi-task learning manner was trained from the dataset and pseudo-labels to learn the PCG representations. Similar methods were adopted for the representation learning of many other kinds of data, such as the ECG and accelerometer data.

In this example, transformations 502 applied to the unlabeled PCG samples include:
Noise addition: Gaussian noise of the same length as the signal and a scale of 0.2 was generated and added to the PCG data.
Scaling: The magnitude of the PCG data was scaled to 80% of the original.
Negation: The PCG data was flipped on the dimension of amplitude.
Flipping: The PCG data was flipped on the dimension of time.
Permutation: The PCG data was cut into ten segments and reconnected in random order.
Stretch: The PCG data was cut into ten segments, and each segment was either stretched or squeezed on the time dimension.

With reference to FIG. 5, there is illustrated a visualization of six categories of transformations 502. During the transformation, each unlabeled original PCG sample was computed to have a 50% possibility of being applied with each kind of transformation. If the sample was applied with a specific type of transformation, a positive pseudo-label was generated under the corresponding task. Otherwise, a negative pseudo-label was generated. At the end of the transformation process, each sample had six binary pseudo-labels, indicating whether the corresponding transformation had occurred.

With reference back to FIG. 4, a 1D CNN derived from the previously mentioned laconic architecture was designed to perform multi-task learning from the transformed PCG dataset and pseudo-labels. This upstream CNN 402 was equipped with the same structure of three convolutional-max pool layers as the downstream model 404. Then the extracted feature vectors were flattened and fed into six branches 406 of fully connected dense layers separately. Each branch was two layers deeper than the section of the downstream model for an easier and quicker converge. Since each task is a binary classification, the last layer was changed to output a pair of possibilities via a SoftMax function.

Preferably, the model may be trained with an Adam optimizer, and the binary cross-entropy may be used to calculate the loss on each task branch. A simple approach was adopted to compute the loss of the upstream network as the sum of the losses for each individual task since all six tasks were derived from pseudo-labels and should be treated equally. The convolutional layers of the upstream model could extract certain representations from PCG data by learning six tasks in parallel.

Referring to FIG. 4, the upstream model 402 is compared with the downstream model 404. After the multi-task model converged in all tasks, the parameters of the first convolutional layer of the upstream model (the θ₁ suggested in equation 2) were transferred to the downstream model as the unchangeable first convolutional layer. The detailed procedure of pre-training upstream model with unlabeled data and generated pseudo-labels and transfer learning is abstracted into pseudocode as presented in the following Algorithm 1—pseudocode for the SSL technique and how it was incorporated into the pipeline to train the efficient 1D-CNN to classify PCG data.

Algorithm 1 The SSL training procedure

```
Input: (X_{L}, Y_{L}): n Labelled data, (X_{L}, Y_{L}) = [x₁^{l}, [symbol]    ), ..., (xₙ^{l}, [symbol]    )];
      X_{U}: m Unlabelled data, X_{U} = [x₁^{u}, ..., xₘ^{u}];
      Mᵤ: Upstream model, Mᵤ(X|Θᵤ^{c}, Θᵤ^{f}) = fₙ(...f₂(f₁(X|θ₁^{c})θ₂^{f})θₙ^{f});
      M_{d}: Downstream model, M_{d}(X|Θ_{d}^{c}, Θ_{d}^{f}) = fₙ(...f₂(f₁(X|θ₁^{c})θ₂^{f})θₙ^{f});
      T = {t¹(X), t²(X), ..., t^{k}(X)}: k upstream tasks;
      x: POG data features;
      y: PCG data labels (5 classes);
      Θ^{c}: Parameters in convolutional layers;
      Θ^{f}: Parameters in fully connected layers;
      f(X|θ): A neural network layer with parameter θ;
Output: M_{d}(X|Θ_{d}^{c}, Θ_{d}^{f}): The trained downstream model;
```

| | |
|---|---|
| 1: | Prepare transfomred data X_{T} = Xᵤ; |
| 2: | Initialize pseudo-labels Y_{P} = [( [symbol] , ..., [symbol] ), ..., ( [symbol] , ..., [symbol] )]=[(0,...),...]; |
| 3: | for all (x,y) in (X_{T},Y_{P}) do |
| 4: | for i in l..k do |
| 5: | Generate a random number r ∈ [0, 1]; |
| 6: | if r ≤ 0.5 then |
| 7: | Apply transfrom to the data x = tⁱ(x); |
| 8: | Annotate transformation y[i] = 1; |
| 9: | end if |
| 10: | end for |
| 11: | end for |
| 12: | Randomly initialize parameters Θᵤ^{c}, Θᵤ^{f} for the upstream model Mᵤ; |
| 13: | repeat |
| 14: | Train upstream model Mᵤ(X\|Θᵤ^{c}, Θᵤ^{f}).fit(Xₜ, Yₚ); |
| 15: | until Number of epochs; |
| 16: | Randomly initialize Θ_{d}^{f} for the downstream model M_{d}; |
| 17: | Transfer convolutional layers' parameters from the upstream model Θ_{d}^{c} = |
| | Θᵤ^{c}; |
| 18: | Freeze parameters θ₁^{c} ⊂ Θ_{d}^{c} in the first convolutional layer ; |
| 19: | repeat |
| 20: | Train downstream model M_{d}(X\|Θ_{d}^{c}, Θ_{d}^{f}).fit(X_{L}, Y_{L}); |
| 21: | until Number of epochs; |

return The trained downstream model Ma(X|0}, ef)

Preferably, the record of sound includes heartbeats, and accordingly heart diseases may be screened based on periodical sounds generated by the heart. Alternatively, breathing sound (i.e., sounds generated in the chest), sound of lung or sound of bowel movement may be analyzed for diagnosis of health issues related other parts of the body, in which the neural network may be trained by different sets of training sets of sounds and diagnostic analysis provided by medical practitioners.

It is appreciated by a person skilled in the art that, even though the classifier or the lightweight network may be designed to analyze sounds generated by a particular organ and generate results based on the classification of sounds generated by that organ, the system is capable of receiving and processing record of sounds generated by multiple organs simultaneously, and selectively mark or classify the components in relation to the sounds generated by a particular organ as desired. It is also possible that in some occasions, an absent of sounds generated by certain organ may also indicate that the user of the app may be suffering from certain diseases.

The inventors performed experimental evaluation and statistical analysis for the system. In the experiment, ten-fold cross-validation was applied in most validation experiments for measuring a model's performance. In each one of the ten iterations of cross-validation, one-fold was set aside as the testing set to evaluate the model trained on the remaining nine folds. Average accuracies from ten iterations are obtained as an aggregated measure from the dataset, taking it as one attempt of 40. Whenever possible, we train neural network models 40 times to account for randomness in model training due to the random initialization of weights, the shuffled order of feeding training samples, and the stochastic optimization. Two-sample unpaired t-tests are used in two-class comparisons.

In some examples, PCG data recorded from devices in the real world may not align with the phases of the cardiac cycle. The inventors devised that a robust model should be able to classify PCG records starting from a random position.

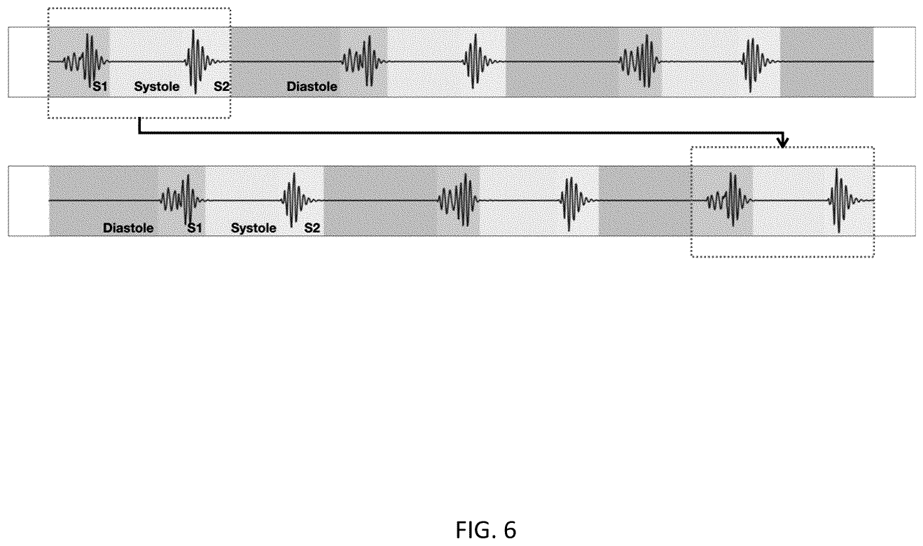

With reference to FIG. 6, to evaluate the robustness of the models, signals from the dataset were perturbated by cutting each recording into two parts at an arbitrary point and swapping the order of two parts. This process was performed before the pre-processing steps mentioned previously. The recording would start from a random position of a cardiac cycle instead of the beginning of S1 after the process. Besides, PCG signals were then polluted in the perturbated dataset with Gaussian noise to further evaluate the robustness of different models. The mean of the normal distribution was maintained as zero while the standard deviation of the distribution was gradually increased from 0.05 to 0.3.

As discussed earlier, with the input record 202 or specifically the recognized audio track 214 after initial validation being processing by the denoising module 206, the health diagnostic analyzing module may be implemented as a lightweight neural network which may run on a computing device with a mobile or lightweight processor. Preferably, the system may be integrated with a function to generate the record of sound being generated by the organ in the animal, either by a built-in microphone or an external microphone connected to the smartphone via any suitable network or connection means.

In one example operation, a patient may launch an app on his smartphone running the health diagnostic system, and start recording his heartbeat by putting the microphone of the smartphone close to his chest proximate to the position of the heart. The system starts with validating the existence of heartbeat, if the validation fails, the app may notify the patient to put the microphone in another position which may help better capturing the sounds of the heart, or until heartbeats are recognized by the app.

Once the validation is successful and the heartbeats of the patient are recognized, the signal is further denoised and then the denoised recording is further analyzed by the lightweight network in the smartphone. The machine learning process may then return to the patient any potential health issue or risk, such as the risk of suffering from VHD, to the patient who perform the diagnosis. The analyzing results, and the recordings may be further provided to a medical consultant for providing additional health advices to the patient.

Preferably, a lightweight deep learning model and a self-supervised learning (SSL) method are provided to utilize unlabeled data to improve the accuracy of valvular heart disease classification using phonocardiogram data. Advantageously, detection and classification of heart murmur using mobile-phone-collected sound may scale-up screening of valvular heart disease at a population level. Highly accurate and lightweight Al models can be deployed in consumer-grade mobile devices may facilitate the adoption of artificial intelligence (AI) methods for mobile health (mHealth) application.

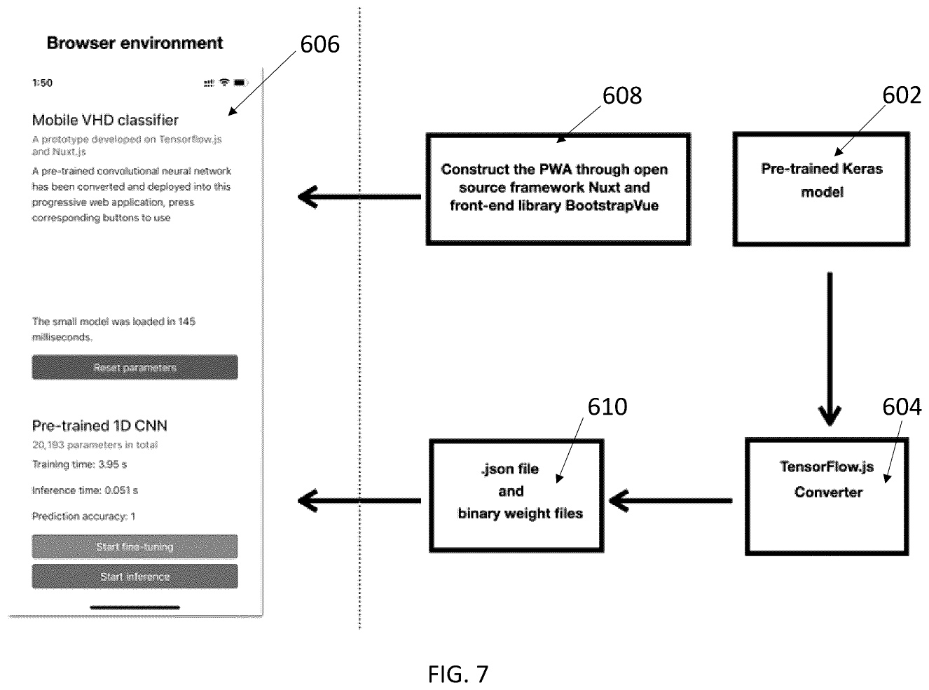

In one example embodiment, the model with pre-trained parameters is deployed to consumer-grade smartphones by converting the KERAS model 602 to TensorFlow.js web-friendly format 604 and developing a progressive web application (PWA) 606 to hold the model behind a basic user interface, referring to FIG. 7. A PWA is a format of application software that may take standards-compliant browsers as the medium and is designed to be capable, reliable and installable to provide an experience that feels like a native application, especially on mobile devices. Advantageously, the relatively easy development, the small installation size and the short launch time of PWA compared with hybrid or interpreted software make it suitable to be involved in academic tasks to gather or distribute information.

Referring to FIG. 7, the user interface of the application and the logical flow behind it were built upon the Nuxt.js framework 608 with the BootstrapVue library, which functionalized the action of fetching model stored in the JSON file and binary weight weights 610 to do fine-tuning or inference on the datasets put in the application. One hundred samples from Yaseen's dataset were held out to perform in-device fine-tuning and inference. The samples were fed into the model with a batch size equal to 2, and 1 epoch of training was conducted in the fine-tuning procedure.

Although the example smartphone-based system was deployed only for demonstrating that the lightweight model can be fine-tuned or make inferences quickly in smartphones, and accuracy was not a major concern, the lightweight 1D CNN was able to achieve 98.65% as the average 10-fold cross-validation accuracy (95% confidence interval (CI: [98.555%, 98.74]), which is better than the accuracy of other example CNN models as shown in the table below. This model's architecture was also applied to the PASCAL dataset, composed of smartphone-recorded recordings. The output layer of the model was modified to fit a binary classification task, but other structures and hyperparameters of the proposed model are maintained. The table below illustrates a comparison of the accuracy and the number of parameters between the present invention and other example models that do not require any prior manual feature extraction or analysis. It is observed that the system according to preferred embodiments of the present invention only consisted of a limited number of parameters while the high accuracy was retained.

| | 10-fold cross- | Number of |
|---|---|---|
| Methods | validation accuracy | parameters |
| CNN + Data augmentation | 98.6% | 279,525 |
| WaveNet | 97% | 320,841 |
| CardioXNet | 99.6% | ~670,000 |
| This Study, lightweight | 98.65% | 20,193 |
| CNN | | |
| This Study, lightweight | 99.435% | 20,193 |
| CNN + SSL | | |

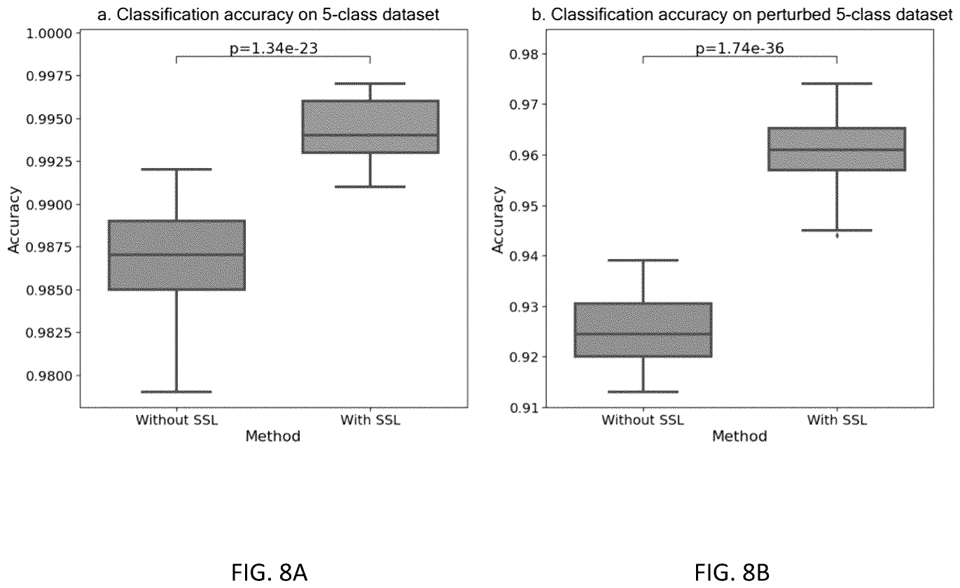

To illustrate the impact of self-supervised learning (SSL), a new upstream model was re-trained to generate a new downstream model to compare the difference in the performance of the model with and without the SSL involved. The 1D CNN with pre-trained parameters achieves an average accuracy of 99.435% (95% CI: [99.380%, 99.495]) by 10-fold cross-validation. The model applied with SSL showed significantly higher accuracy in classifying five categories of PCG data as shown in FIG. 8A.

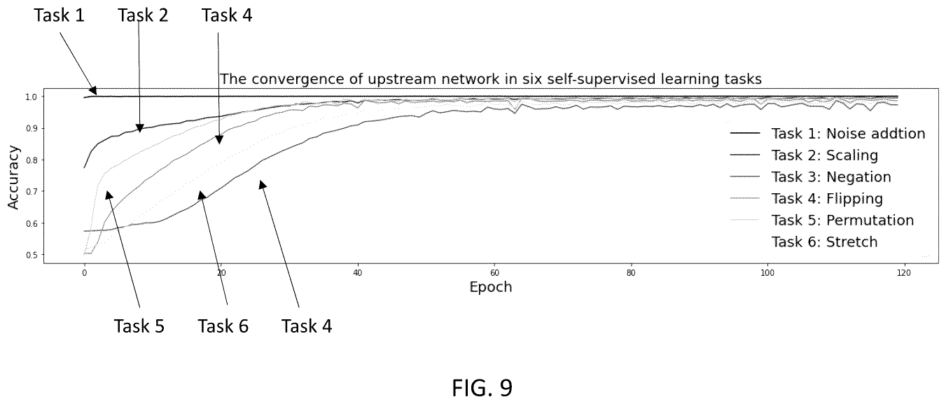

During the upstream SSL, the upstream networks can converge in all six tasks after a certain number of epochs of learning to capture basic representations from PCG data. The training curves for six tasks in the upstream network are presented in FIG. 9.

To further verify the improvements brought by SSL, the same pipeline was applied to the train-test-split PASCAL dataset to see whether the testing accuracy could be significantly improved after incorporating pre-trained parameters into the downstream model. The average testing accuracies of the model with and without SSL are 79.78% (95% CI: [78.348%, 81.211%]) and 74.19% (95% CI: [72.014%, 76.369%]), respectively. These results show that SSL significantly increases the prediction accuracy of the model (p-value=4.27×10⁻⁵). The relatively low-test accuracy of the PASCAL dataset is likely caused by the limited amount of training data and lack of preprocessing, such as segmentation and denoising that are available in the dataset. Nonetheless, the result demonstrates that SSL is indeed useful in improving the accuracy of the proposed lightweight CNN.

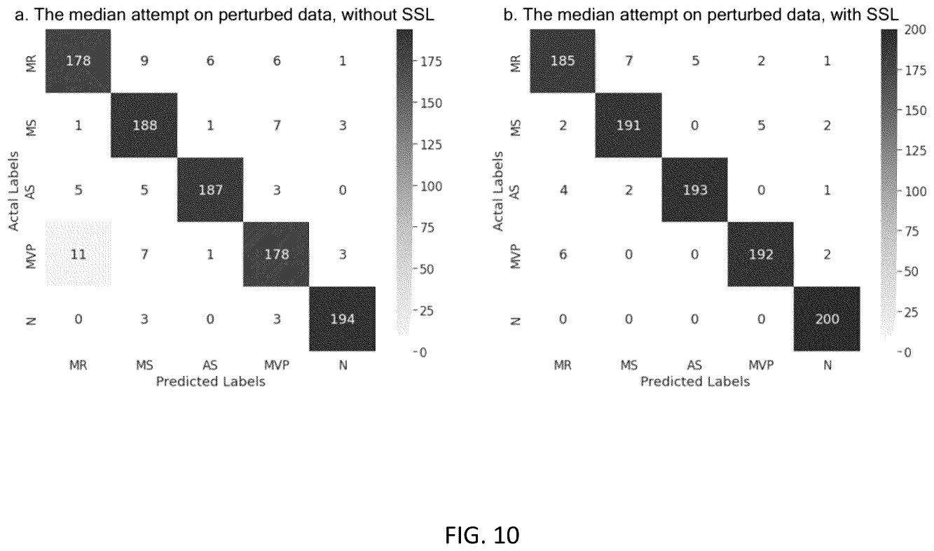

Training and testing were performed on perturbed data through the model in both cases with and without SSL. An even more compelling gap between the performance of the two methods is demonstrated when the data becomes less formatted. The average 10-fold cross-validation accuracy of the model without SSL drops to 92.538% (95% CI: [92.304%, 92.771%]). The model with SSL shows better robustness, and the number decreased to 96.123% (95% CI: [95.911%, 96.334%]). The summary of all attempts is plotted in the box chart from FIG. 8B. The confusion matrices of the median attempts from both methods are shown in FIG. 10. The model without SSL performed notably worse when the data had random starting points. Specifically, the accuracy of recognizing MR and MVP categories falls below 90%. Alternatively, the model with SSL is more robust and less affected by the reformation of the data.

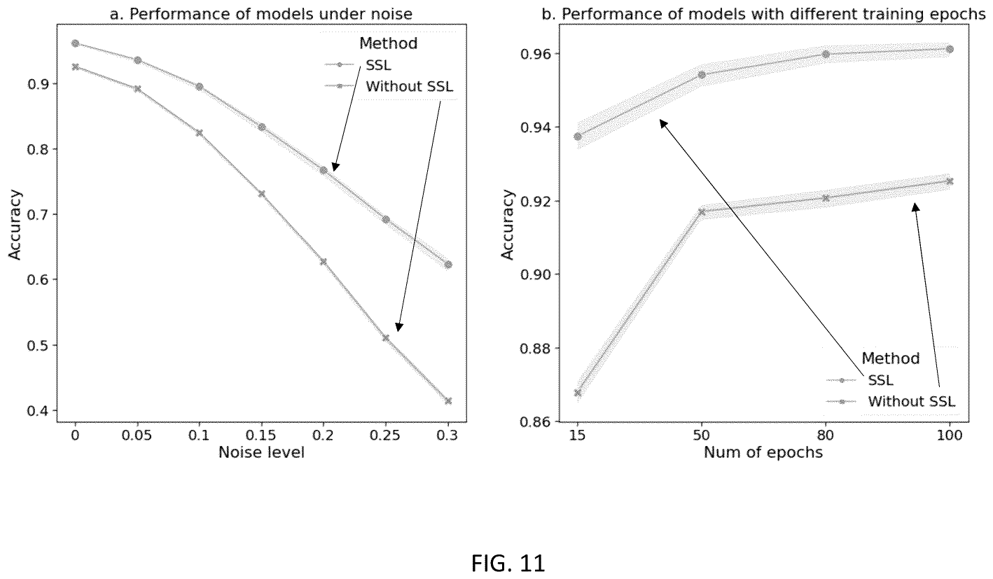

The experiments comparing models with and without SSL on the reformatted dataset with different gaussian noise levels are plotted in the line chart in FIG. 11A. With the help of unlabeled data, the model consistently outperformed the model that was purely trained with labelled data. Moreover, a growing gap between the accuracy of the two models can be witnessed with the increase in noise level. In addition, the model converged much faster when the first convolutional layer was pre-trained with unlabeled data. Referring to FIG. 11B, the model with SSL had already achieved higher accuracy than the model without SSL under 100 epochs of training when only 15 epochs of training were conducted.

In another experiment carried out in an evaluation study, PWAs on four different smartphones were installed with two mainstream operating systems, i.e., iOS and Android, to evaluate the time needed to tune or execute the proposed lightweight model. Another model with more filters, larger kernels in convolutional layers, and a deeper section of fully connected dense layers was also constructed and deployed to the application for comparison. The large-size model contained 241,597 parameters. The following table shows that using 100 samples to fine-tune the small model completes in 5 seconds on two iOS devices that cooperated with Safari browsers. The inference on 100 samples accomplishes immediately within tens of milliseconds. However, the larger model in iPhones takes nearly double the time to perform the fine-tuning, and the inference time of the two models differed by orders of magnitude. There are similar gaps in the performance of the two models on Android devices with Firefox browsers, and the operation speeds vary dramatically based on hardware differences. The additional running time caused by the increased number of parameters affects the implementation of the model in a resource-limited device. For example, the large model requires more than 5 seconds to complete the inference in Samsung Galaxy A30, while the small model only needs less than 0.4 seconds.

With reference to the table below, six example smartphones installed with two mainstream operating systems were used to run the app. The processing time recorded in the table is an average out of five attempts.

| | OS platform | Small-size model | | | Large-size model | | |
|---|---|---|---|---|---|---|---|
| Model | and browser | Fine-tuning | | Inference | Fine-tuning | | Inference |
| iPhone 14 | iOS, Safari | 4.537 | s | 0.030 s | 8.329 | s | 0.223 s |
| Pro Max | | | | | | | |
| iPhone 12 | iOS, Safari | 3.922 | s | 0.054 s | 9.487 | s | 0.342 s |
| iPhone SE | iOS, Safari | 4.492 | s | 0.059 s | 10.087 | s | 0.497 s |
| Samsung | Android, Firefox | 28.917 | s | 0.367 s | 65.604 | s | 5.665 s |
| Galaxy A30 | | | | | | | |
| Xiaomi 13 | Android, Firefox | 1.946 | s | 0.058 s | 2.696 | s | 0.605 s |
| Xiaomi 11 | Android, Firefox | 2.924 | s | 0.060 s | 3.773 | s | 0.552 s |

TensorFlow.js leverages the WebGL API as a backend to enable efficient GPU-accelerated training and inferencing of deep learning models. However, it has been observed that the maximum supported texture size varies across different browsers and operating systems, which can impact a model's ability to be executed successfully on a given device. Notably, Chrome, which is the current most dominant browser on mobile devices, only supports a maximum texture size of 4096 in tested Android devices and attempts to execute a large-size model on three tested Android devices using Chrome browser failed due to the model's texture size exceeding WebGL's maximum.

It was observed that the deep learning models may not be executed successfully in Chrome browser on Android devices due to WebGL's limitations and the fact that the large-size model in this section has fewer parameters than other models. However, on iOS and other Android browsers, the maximum accepted texture size is higher, enabling successful execution of the larger model, albeit with longer processing times.

Removing the redundant parameters in deep neural networks reduces the time required to perform calculations and considerably saves the costs of energy consumption. The power consumption of executing the small and large models was measured and compared on an average smartphone. Three PWAs were deployed to have 1) the user interface only, 2) the interface plus performing inference ten times by the small-size model and 3) the interface plus performing inference ten times by the large model, respectively. Battery consumptions for three PWAs were analyzed by Greenspector on a Galaxy S7 with Android 8 installed on a standardized visit. The detailed analysis methodology was given on Greenspector's website. Five attempts of measurement were recorded. The energy consumption is nearly tripled for the PWA running on the large-size model compared to the small-size model, from around 0.6 mAh to 1.7 mAh, which is a statistically significant increase (t-test, p=1.19×10⁻⁷).

Advantageously, the lightweight model's effective and efficient execution of fine-tuning and inference on mobile devices illustrates that it is feasible to provide a classification of PCG data through deep learning models in a distributed computing paradigm without the need to transfer data and the dependence on a central server. Furthermore, the model can be fine-tuned locally in a reasonable period. It is possible to apply recent decentralized machine learning training techniques like federated learning and swarm learning so that more data can be involved by aggregating updated parameters from edge devices, or a more personalized classification can be provided.

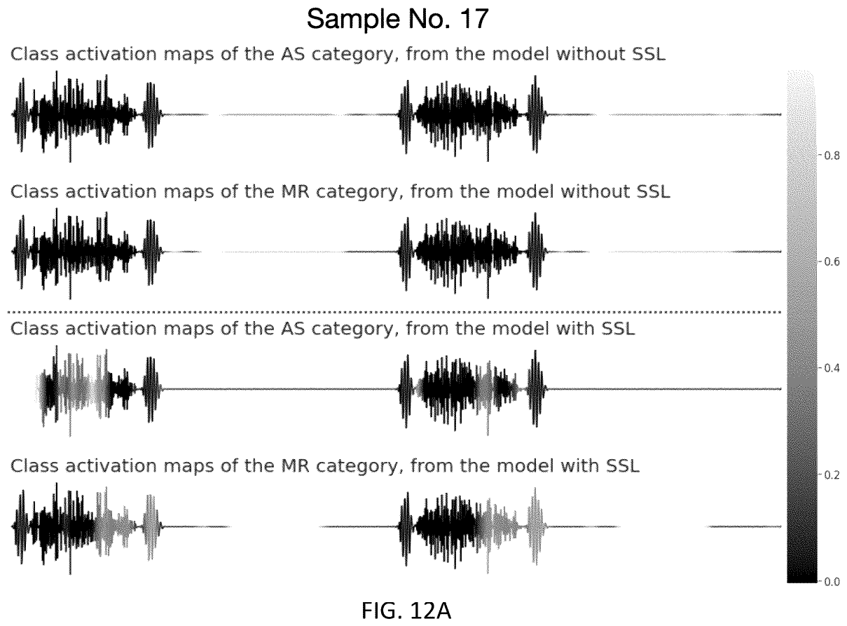

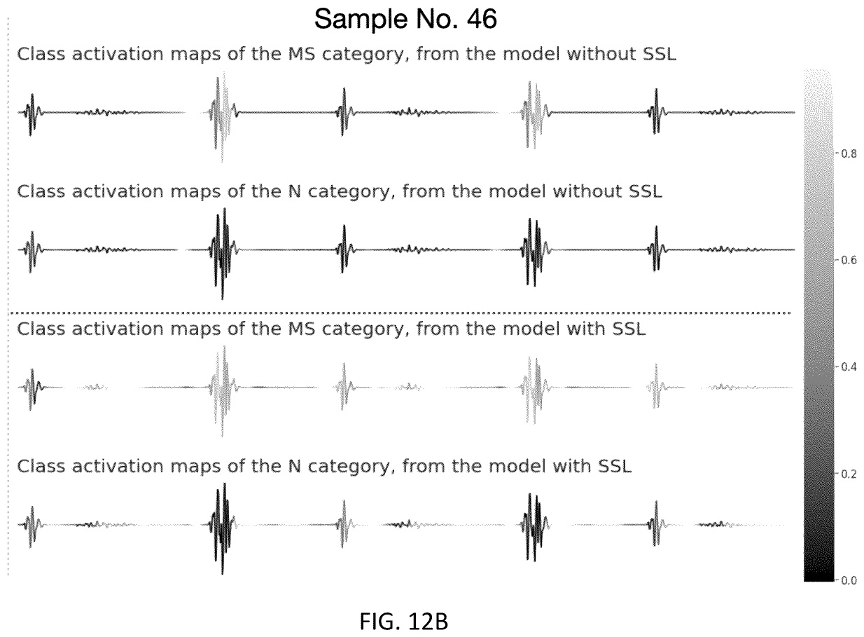

In yet another experiment performed by the inventors, class activation maps (CAM) of both the actual category and predicted category produced by GradCAM++ were generated from the convolutional layers of representative CNN trained with SSL and without SSL. The purpose of this experiment is to visualize the important features that contribute to the prediction. With referent to FIGS. 12A and 12B, two soundtracks, namely No. 17 and No. 46, are selected as case studies because they are misclassified by the model without SSL but correctly classified by the model with SSL. The actual label for Sample No. 17 is AS, but the model without SSL misclassified it as MR.

As presented from the CAM, the activated parts of the AS category from the non-SSL model are similar to those of the MR category. Meanwhile, most attention is drawn to the diastolic stage instead of the systolic stage, where the murmur is located. In contrast, the activated regions of the actual class from the SSL model cover the most systolic stage of the sample, which ensures that the important characteristics of the murmur of aortic stenosis are well captured (i.e., the murmur is mid-systolic, diamond-shaped, and high-pitched).

A similar situation was observed in Sample No. 46. The murmur in the diastole was not noticed by the convolutional layers in the non-SSL model, as demonstrated by the CAM. This neglect of murmur gave the model the wrong judgement. In opposition, the model with SSL involved a more extensive range of activated areas of the MS category, including S1, S2 and the diastolic murmur, which drove the model to identify murmurs and make the proper judgement about the position of the murmur.

With the facilitation of SSL and massive unlabeled data, the lightweight neural network may provide a rapid, accurate and robust classification of the five-class PCG dataset, even on consumer-grade smartphones with relatively limited computing resources.

The inventors devised that, outside of laboratory conditions, heart sounds recorded by mobile devices may be weak, noisy, and chaotic in stages. Although SSL is tested to improve the robustness of PCG classification, provision may be made from two aspects for the future real-world scenario of mobile phone-based VHD automatic screening.

Advantageously, efficient heart sound denoising and segmentation algorithms have been developed to process the real-world raw PCG signal, and optionally, signal amplification or augmentation procedures may also be applied to leverage differences between mobile phones and digital stethoscopes.

Preferably, in an alternative embodiment, tailor-made SSL tasks for PCG data may also be developed, while upstream contrastive tasks used in this study were universally applied to other biomedical signals. It is devised that design patterns balancing the upstream and the downstream model can be further tuned/optimized. For example, transferring and freezing more than one convolutional layer of parameters to the downstream model leads to a performance decrement. Setting a large number of unlearnable parameters may tighten convolutional layers from the upstream task and may cause the loss of focus from the downstream task in the small model used in the present study.

In addition, a phone-recorded PCG database can be established to cope with the phone-based VHD screening scenario in the real world.

These embodiments may be advantageous in that, a lightweight 1D CNN is provided. The system achieves an average accuracy of 98.6% in 10-fold cross-validation in a five-class PCG dataset. The neural network has a 92%-97% reduction of parameters compared to other example deep learning models.

Advantageously, the present invention significantly improves the classification average accuracy to 99.4%, as well as the robustness of the model by involving around 25,000 unlabeled data from PhysioNet and performing contrastive self-supervised learning. In addition, the model may be converted to a browser-executable version and deployed to an application to conduct instant PCG classification on smartphones. The condition of being a lightweight model allows it to be deployed in more smartphones. It also can reduce power consumption.

In addition, the class activation maps are generated and compared to show that models with pre-trained layers can better capture discriminatory features with biological relevance. Early screening for VHD is an important medical topic, and the present invention may enhance the efficiency of deep learning-based screening for VHD with the help of unlabeled data and blend reliable PCG classification into mobile health.

As described earlier, sound of lung may be analyzed for diagnosis of health issues related other parts of the body, i.e., lung diseases. The inventors devised an alternative embodiment of the health diagnostic system, in which the inventors called it “Ausculto-Lung”, and may be used for to detect abnormalities in lung sounds and screen for airway-related diseases.

Lung diseases are a significant cause of morbidity and mortality worldwide. The early detection and diagnosis of respiratory abnormalities are essential for effective management and treatment of lung diseases. Auscultation is a non-invasive technique that involves listening to the sounds generated by the lungs during breathing. The inventors devised that by employing Al techniques, the process of lung sound analysis may be automated and the accuracy and reliability of the diagnosis may be improved.

In one example embodiment, Ausculto-Lung may consist of three main parts: recognition, denoising, and classification.

Preferably, the recognition phase involves the identification and location of lung sounds to ensure the quality of captured data. Recordings are downsampled to 1.6 KHz and windowed into 2-second clips, which is different from heartbeat detection where recordings of 1-second clips are processed. Each clip may preferably contain a complete breathing cycle or part of the cycle may also be sufficient for some screen process. Urban noises and generated Gaussian noises are regarded as positive data, and a convolutional neural network (CNN) of similar architecture to the heart sound recognition model may be used, with the input layer being different.

The denoising phase may involve the removal of background noise and other artifacts that interfere with the quality of the captured lung sounds, similar to the process in VHD screening. The procedure is similar to the denoising procedure in heart sound analysis; however, the boundary may be set to 800 Hz instead of 300 Hz based on the characteristics of the lung sound data.

The classification phase may involve the classification of lung sounds into normal, crackles, or wheezes. Recordings may be downsampled to 1.6 KHz and chopped into breathing cycles. For example, each cycle may be cropped or padded into 3 seconds long, with the average length of a cycle in the training set being around 2.8 seconds. A CNN of similar architecture to the heart sound classification model may be used, with the input layer being different, to classify cycles into normal/contain crackle/contain wheeze. Preferably, hyper-parameter tuning may be employed to improve the accuracy.

The inventors also tested the embodiments of Ausculto-Lung, using the Respiratory Sound Database, which contains 920 recordings from 126 patients, with 6898 respiratory cycles. Of these, 1864 contain crackles, 886 contain wheezes, and 506 contain both crackles and wheezes. The recordings from 26 patients form the testing set, while the recordings from the other 100 patients form the training set. It was observed that the testing accuracy for the recognition phase is 91.10%.

Advantageously, Ausculto-Lung may be used for detecting respiratory abnormalities and screening for airway-related diseases. It can be used to complement the traditional auscultation technique and improve the accuracy and reliability of the diagnosis. Moreover, it can be used to monitor the progression of lung diseases and adjust the treatment plan accordingly.

Although not required, the embodiments described with reference to the figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilized. This will include tablet computers, wearable devices, smart phones, Internet of Things (IoT) devices, edge computing devices, stand-alone computers, network computers, cloud-based computing devices and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A health diagnostic system, comprising:
a signal receiver module arranged to receive a record of sound generated by an organ in a body of an animal when the organ performs a predetermined function for a predetermined period;
a signal denoising module arranged to reduce a noise signal in the record; and
a health diagnostic analyzing module arranged to analyze a health issue of the animal based on normal sounds and adventitious sounds generated by the organ in the denoised record.

2. The health diagnostic system of claim 1, wherein the adventitious sounds include murmur and an arrythmia sound pattern generated by the heart of the animal.

3. The health diagnostic system of claim 2, wherein the health diagnostic analyzing module includes a neural network arranged to classify the record so as to mark at least one health indicator associated with the health issue.

4. The health diagnostic system of claim 3, wherein the denoising module is arranged to reduce a disturbance caused by the noise signal in the record so as to increase a prediction accuracy of the health issue provided by the health diagnostic analysing module.

5. The health diagnostic system of claim 4, wherein the neural network includes a lightweight neural network arranged to run on a computing device with a mobile or lightweight processor.

6. The health diagnostic system of claim 1, wherein the signal receiver module is further arranged to validate the normal sounds generated by one or more organs in the body of the animal for further process.

7. The health diagnostic system of claim 6, wherein the signal receiver module comprises a signal recognition module arranged to validate the normal sounds by recognizing an existence of the sounds in the record received by the signal receiver module; and wherein the signal denoising module and the health diagnostic analyzing module are arranged to process the record of sound upon successful validation of normal sounds in the record.

8. The health diagnostic system of claim 7, wherein the record includes one or more clips extracted from the record received by the signal receiver module.

9. The health diagnostic system of claim 7, wherein the signal receiver module is further arranged to prolong the record of sound for further process upon successful validation of the normal sounds.

10. The health diagnostic system of claim 9, wherein the successful validation is indicated by positive classification of two consecutive clips, each containing the normal sounds.

11. The health diagnostic system of claim 5, wherein the health issue includes a risk of valvular heart disease (VHD).

12. The health diagnostic system of claim 11, wherein the neural network comprises a downstream phonocardiogram (PCG) classification network for VHD screening.

13. The health diagnostic system of claim 12, wherein the downstream PCG classification network is trained based on an upstream self-supervised learning network for PCG classification and a transfer learning process.

14. The health diagnostic system of claim 13, wherein the health diagnostic analyzing module is arranged to label a phonocardiogram associated with the record received by the signal receiver, thereby facilitating the downstream PCG classification network to mark the at least one health indicator associated with the health issue.

15. The health diagnostic system of claim 1, wherein the record of sound includes heartbeats, breathing sound, sound of lung or sound of bowel movement.

16. The health diagnostic system of claim 1, wherein the signal receiver module comprises a microphone arranged to generate the record of sound generated by one or more organs in the animal.

17. A method for analyzing health of an animal, comprising the steps of:
receiving a record of sound generated by an organ in a body of the animal when the organ performs a predetermined function for a predetermined period;
reducing a noise signal in the record; and
analyzing a health issue of the animal based on normal sounds and adventitious sounds generated by the organ in the denoised record.

18. The method of claim 17, wherein the step of analyzing the health issue of the animal based on normal sounds and adventitious sounds in the denoised record includes classify the record by a neural network so as to mark at least one health indicator associated with the health issue.

19. The method of claim 18, wherein the step of reducing a noise signal in the record is performed to reduce a disturbance caused by the noise signal in the record so as to increase a prediction accuracy of the health issue being analyzed.

20. The method of claim 17, further comprising the step of validating the normal sounds generated by one or more organs in the body of the animal for further process, by recognizing an existence of the sounds in the record received, prior to the steps reducing a noise signal in the record and the step of analyzing a health issue of the animal based on normal sounds and adventitious sounds generated by the organ in the denoised record.
